(19) United States
(12) Patent Application Publication
(10) Pub. No.: US 2024/0225990 A9
(43) Pub. Date: Jul. 11, 2024

(54) PRODUCTION PROCESS OF A CONVENIENT AND QUICK-ADHESIVE EYELASHES

(71) Applicant: Qingdao Homay Eyelashes Co.,Ltd., Qingdao (CN)

(72) Inventor: MINGTAO ZHANG, Qingdao (CN)

(21) Appl. No.: 17/978,667

(22) Filed: Nov. 1, 2022

(30) Foreign Application Priority Data
Oct. 24, 2022 (CN) 202211300975.7

(51) Int. Cl. A61K 8/81; A61K 8/22; A61K 8/34; A61K 8/36

(52) U.S. Cl. CPC A61K 8/8152; A61K 8/22; A61K 8/345; A61K 8/347; A61K 8/36; A61K 2800/54

(57) ABSTRACT

The present application discloses a production process of a convenient and quick-adhesive eyelashes in the technical field of daily necessities, and adopts the following technical solution: comprising the following steps: applying a certain amount of glue to front or back of the root of the grafting eyelashes in direction from root to tip of the hair, the glue consists of the following components: mass percentage content: 37.8-48% isooctyl acrylate; 11.9-13% butyl methacrylate; 6-7% n-butyl acrylate; 0.9-1.5% methyl methacrylate; 0.08-0.2% ethylene glycol diacrylate; 31-42% water; 0.3-1% propylene glycol; 0.07-0.13% tert-butyl peroxide; 0.05-0.15% sodium formaldehyde sulfoxylate; 0.05-0.1% sodium alkyl benzene sulfonate; 0.05-0.1% nonylphenol ethoxylate; 0.04-0.1% DCOIT; 0.1-0.2% ammonium persulfate; 0.05-0.15% sodium bicarbonate and 0.2-2% carbon black, after applying a certain amount of glue, let it dry at room temperature for 5 minutes-2 hours before packaging and sterilization and leave the factory for use.

### CROSS-REFERENCE TO RELATED APPLICATIONS

This application claims the benefit and priority of Chinese patent application No. 202211300975.7, filed on Oct. 24, 2022, disclosure of which is hereby incorporated by reference in its entirety.

### TECHNICAL FIELD

The present application relates to the technical field of daily necessities, and in particular, to a production process for a convenient and quick-adhesive eyelashes.

### BACKGROUND

At present, when grafting eyelashes are mentioned in the world, many consumers still think that grafting false eyelashes will only be available in beauty salons and nail salons, which requires professional operation. Improper operation can be very dangerous. For example: 1. If you operate it yourself, the tweezers will scratch the eyes and surrounding skin, and it is a waste of time and inconvenient. Professionals must be very careful when operating; 2. Almost all the grafting glues on the market are chemical quick-drying, similar to the 502 glues on the market. Once it touches the skin or eyes, improper use will lead to serious consequences. With the rise of grafting false eyelashes, various industrial glues have appeared in the eyelash market. Consumers also follow the old methods when using them. They either go to the eyelash store for grafting, or use tweezers to clamp the eyelashes themselves, and then dip the commonly used glue on the market. (Industrial Chemical Adhesive, Pressure Sensitive Adhesive, Constant Speed Drying Adhesive or Self Adhesive). In these glues, the quick-drying glue is easy to solidify and seal the hair scales of the original eyelashes, and it is difficult to fall off, which is very harmful to the consumer's own eyelashes, which can easily cause their own eyelashes to fall off and affect the regeneration of eyelashes. If the chemical glue such as pressure-sensitive adhesive dipped in, starts to be grafting after the glue is semi-dry or a few seconds after dipping, the liquid or semi-solid (paste) glue will fully bond to your false eyelashes. When removing makeup and cleaning, it is extremely laborious, and it is not easy to fall off and stick to your eyelashes. If you use cleansers, be extra careful not to drip or spill into your eyes, which can burn and cause inflammation.

To sum up: there are the following disadvantages:
1. It is technically difficult and inconvenient to operate and wear individual self-grafting false eyelashes;
2. The existing glues are quick-drying and non-drying: the quick-drying type has great harm to the consumer's own eyelashes, and the quick-drying glue requires professional operation, which is time-consuming, labor-intensive, expensive, and dangerous. Any spillage of glue on the eyes or surrounding skin can cause blindness or skin injury. The use of self-adhesive glue is cumbersome. Dip or smear it on the grafting false eyelashes, wear it directly or wear it directly after tens of seconds. It is easy to loose and fall off, and it is difficult to clean up when removing makeup and cleaning the glue. When wearing it, you have to wait for the glue to fully solidify, which is a waste of time. The use of self-adhesive glue is cumbersome. Dip or smear it on the grafting false eyelashes, wear it directly or wear it directly after tens of seconds. It is easy to loose and fall off, and it is difficult to clean up when removing makeup and cleaning the glue. When wearing it, you have to wait for the glue to fully solidify, which is a waste of time And the viscosity is not high and cannot be used repeatedly. After boiling and sterilizing at high temperature, ordinary glue cannot be used repeatedly. After a long time, ordinary glue is easy to dry, crack and harden, and its viscosity is greatly reduced. There are great safety hazards and inconvenient cleaning, as well as many inconvenient factors such as time wasting, high cost, and location and store restrictions.

### SUMMARY

The purpose of this section is to outline some aspects of the embodiments of the present application and to briefly introduce some preferred embodiments. Some simplifications or omissions may be made in this section and in the abstract and application title of this application to avoid obscuring the purpose of this section, abstract and application title, and such simplifications or omissions should not be used to limit the scope of this application.

The present application is proposed in view of the above-mentioned problems in the existing production process of a convenient and quick-adhesive type of eyelashes.

Therefore, the purpose of the present application is to provide a production process for a convenient and quick-adhesive eyelashes, which enables consumers who like to graft false eyelashes to be at home (or anywhere), faster, easier, safer, and more cost-effective. It is more convenient to complete the grafting by yourself, including the quick wearing of a single pair of eyelashes, and the self-developed glue can also be quickly worn and convenient.

In order to solve the above-mentioned technical problems, the application provides a production process of a convenient and quick-adhesive eyelashes, comprising the following steps:
applying a certain amount of glue to front or back of the root of the grafting eyelashes in direction from root to tip of the hair,
the glue consists of the following components: mass percentage content: 37.8-48% isooctyl acrylate; 11.9-13% butyl methacrylate; 6-7% n-butyl acrylate; 0.9-1.5% methyl methacrylate; 0.08-0.2% ethylene glycol diacrylate; 31-42% water; 0.3-1% propylene glycol; 0.07-0.13% tort-butyl peroxide; 0.05-0.15% sodium formaldehyde sulfoxylate; 0.05-0.1% sodium alkyl benzene sulfonate; 0.05-0.1% nonylphenol ethoxylate; 0.04-0.1% DCOIT; 0.1-0.2% ammonium persulfate; 0.05-0.15% sodium bicarbonate and 0.2.2% carbon black.

Optionally, the types of the grafting eyelashes include single eyelashes, multiple eyelashes, single cluster eyelashes, hot-melt eyelashes at the root, glued eyelashes at the root, segmented eyelashes, single eyelashes, A-type eyelashes, fishtail eyelashes, lower eyelashes, single pair of eyelashes, single pair of lower eyelashes.

Optionally, the material of the grafting eyelashes is animal hair, human hair, degradable silk, chemical fiber silk or protein silk.

Optionally, the grafting eyelashes are a single pair of false eyelashes or a single pair of lower eyelashes, and a certain amount of glue is applied on root eyelash stems of the single pair of false eyelashes or the single pair of lower eyelashes, and an applied glue height W is 1-2 mm, and a thickness H is 0.1-1 mm, a width L is determined according to a width of the root of the eyelashes.

Optionally; the height W of the glue applied to the root of the grafting eyelashes is 1-3 mm, the thickness H is 0.1-1 mm, and the width L is determined according to the width of the root of the eyelashes.

Optionally, after applying a certain amount of glue, let it dry at room temperature for 5 minutes-2 hours before packaging and sterilization and leave the factory for use.

Optionally, a small brush or a glue dispenser tool is used to apply the glue.

Optionally, the glue consists of the following components: mass percentage content: 37.8-38.3% isooctyl acrylate; 11.9-12.4% butyl methacrylate; 6.1-6.6% n-butyl acrylate; 0.9-1.3% methyl methacrylate; 0.08-0.17% ethylene glycol diacrylate; 41.4-41.9% water; 0.3-0.8% propylene glycol; 0.07-0.13% Cert-butyl peroxide; 0.05-0.15% sodium formaldehyde sulfoxylate; 0.05-0.1% sodium alkyl benzene sulfonate; 0.05-0.1% nonylphenol ethoxylate; 0.04-0.09% DCOIT; 0.1-0.2% ammonium persulfate; 0.05-0.15% sodium bicarbonate and 0.2-2% carbon black.

Optionally, the glue consists of the following components: mass percentage content: 38-48% isooctyl acrylate; 1243% butyl methacrylate; 6-7% n-butyl acrylate; 0.9-1.5% methyl methacrylate; 0.08-0.2% ethylene glycol diacrylate; 31-42% water; 0.3-1% propylene glycol; 0.07-0.13% tert-butyl peroxide; 0.05-0.15% sodium formaldehyde sulfoxylate; 0.05-0.1% sodium alkyl benzene sulfonate; 0.05-0.1% nonylphenol ethoxylate; 0.05-0.1% DCOIT; 0.1-0.2% ammonium persulfate; 0.05-0.15% sodium bicarbonate and 0.2-2% carbon black.

To sum up, the present application includes at least one of the following beneficial effects:
1. The wearing of grafting eyelashes, including a single pair of false eyelashes, is faster, safer and more convenient.
2. To solve the problem of glue, use the latest self-developed non-drying glue, and apply the glue to the root of the eyelashes in advance, which is convenient for fast wearing and easy to remove makeup. The finished eyelashes produced are resistant to high temperature of 80-100 degrees, and the boiling water disinfection will not affect the use effect. It surpasses the false eyelash glue currently on the market in terms of application and adhesion performance and stretch resilience, and can be used repeatedly.
3. After applying the glue, all kinds of eyelashes are not only fast, safe and easy to remove makeup, but also can be worn repeatedly to increase the frequency of use and improve the cost performance.
4. After a long time, it is not easy to dry and crack and the viscosity does not decrease, keeping it soft and comfortable.

### BRIEF DESCRIPTION OF DRAWINGS

In order to illustrate the technical solutions of the embodiments of the present application more clearly, the following briefly introduces the accompanying drawings used in the description of the embodiments. Obviously, the drawings in the following description are only some embodiments of the present application. For those of ordinary skill in the art, other drawings can also be obtained from these drawings without any creative effort.

FIG. 1 is a schematic structural diagram of grafting segmented eyelashes according to the present application;

FIG. 2 is a schematic structural diagram of grafting fishtail type eyelashes according to the present application;

FIG. 3 is a schematic structural diagram of grafting single pair of eyelashes according to the present application.

### REFERENCE SIGNS

1. Grafting eyelashes; 2. Glue.

### DETAILED DESCRIPTION

The present application will be further described in detail below conjunction with accompanying drawings 1-3.

### Embodiment 1

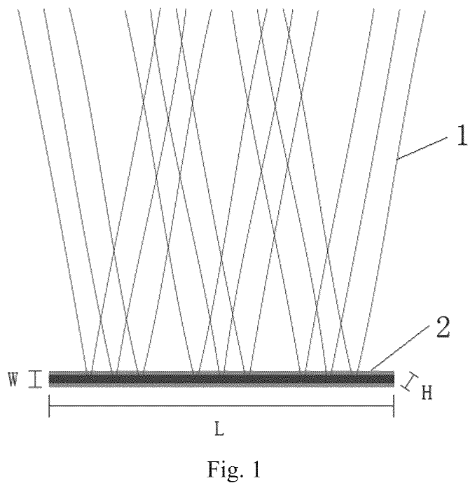

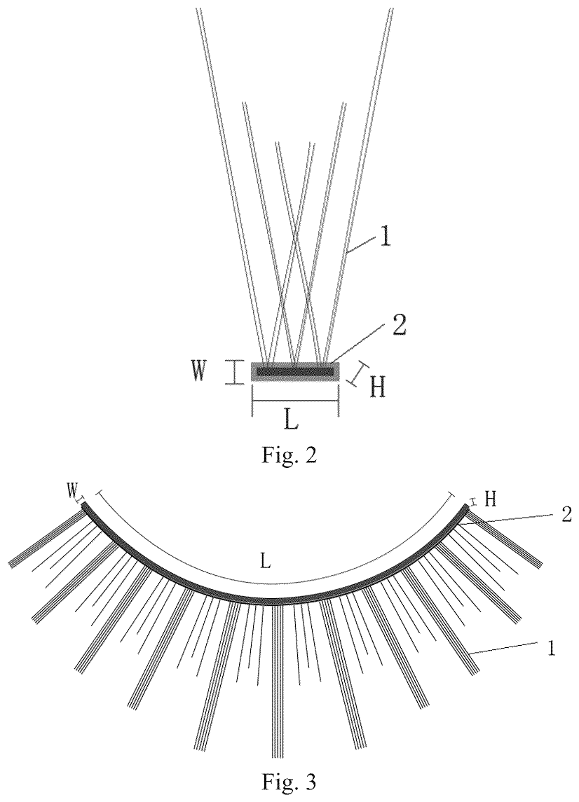

Referring to FIGS. 1-2, the present application discloses a production process for a convenient and quick-adhesive eyelashes, comprising the following steps: applying a certain, amount of glue 2 to front or back of the root of the segmented eyelashes or fishtail eyelashes, which is made of animal hair, human hair, degradable silk, chemical fiber silk or protein silk, in direction from root to tip of the hair by a small brush or a glue dispenser tool. The height W of the glue 2 applied to the root of the eyelashes is 1-3 mm, the thickness H is 0.1-1 min, and the width L is determined according to the width of the root of the eyelashes.

The glue 2 consists of the following components: mass percentage content: 37.8-48% isooctyl acrylate; 11.9-13% butyl methacrylate; 6-7% n-butyl acrylate; 0.9-1.5% methyl methacrylate; 0.08-0.2% ethylene glycol diacrylate; 31-42% water; 0.3-1% propylene glycol; 0.07-0.13% tert-butyl peroxide; 0.05-0.15% sodium formaldehyde sulfoxylate; 0.0540.1% sodium alkyl benzene sulfonate; 0.05-0.1% nonylphenol ethoxylate; 0.04-0.1% DCOIT; 0.1-0.2% ammonium persulfate; 0.05-015% sodium bicarbonate and 0.2-2% carbon black.

### Embodiment 2

Referring to FIGS. 1-2, the present application further discloses a production process for a convenient and quick-adhesive eyelashes, comprising the following steps: applying a certain amount of glue 2 to front or back of the root of the segmented eyelashes or fishtail eyelashes, which is made of animal hair, human hair, degradable silk, chemical fiber silk or protein silk, in direction from root to tip of the hair by a small brush or a glue dispenser tool. The height W of the glue 2 applied to the root of the eyelashes is 1-31 mm, the thickness H is 0.1-1 mm, and the width L is determined according to the width of the root of the eyelashes.

The glue 2 consists of the following components: mass percentage content: 38-48% isooctyl acrylate; 12-13% butyl methacrylate; 6-7% n-butyl acrylate; 0.9-1.5% methyl methacrylate; 0.08-0.2% ethylene glycol diacrylate; 31-42% water; 0.3-1% propylene glycol; 0.07-0.13% tert-butyl peroxide; 0.05-0.15% sodium formaldehyde sulfoxylate; 0.05-0.1% sodium alkyl benzene sulfonate; 0.05-0.1% nonylphenol ethoxylate; 0.05-0.1% DCOIT; 0.1-0.2% ammonium persulfate; 0.05-0.15% sodium bicarbonate and 0.2-2% carbon black.

### Embodiment 3

Referring to FIG. 3, the present application discloses a production process for a convenient and quick-adhesive eyelashes, comprising the following steps: applying a certain amount of glue 2 to front or back of the root of the segmented eyelashes or fishtail eyelashes, which is made of animal hair, human hair, degradable silk, chemical fiber silk or protein silk, in direction from root to tip of the hair by a small brush or a glue dispenser tool. The height W of the glue 2 applied to the root of the eyelashes is 1-2 mm, the thickness H is 0.1-1 mm, and the width L is determined according to the width of the root of the eyelashes.

The glue 2 consists of the following components: mass percentage content: 37.8-38.3% isooctyl acrylate; 11.9-12.4% butyl methacrylate; 6.1-6.6% n-butyl acrylate; 0.9-1.3% methyl methacrylate; 0.08-0.17% ethylene glycol diacrylate; 41.4-41.9% water; 0.3-0.8% propylene glycol; 0.07-0.13% tert-butyl peroxide; 0.05-0.15% sodium formaldehyde sulfoxylate; 0.05-0.1% sodium alkyl benzene sulfonate; 0.05-0.1% nonylphenol ethoxylate; 0.04-0.09% DCOIT; 0.1-0.2% ammonium persulfate; 0.05-0.15% sodium bicarbonate and 0.2-2% carbon black.

### Embodiment 4

Referring to FIG. 3, the present application discloses a production process for a convenient and quick-adhesive eyelashes, comprising the following steps: applying a certain amount of glue 2 to front or back of the root of the segmented eyelashes or fishtail eyelashes, which is made of animal hair, human hair, degradable silk, chemical fiber silk or protein silk, in direction from root to tip of the hair by a small brush or a glue dispenser tool. The height W of the glue 2 applied to the root of the eyelashes is 1-2 mm, the thickness H is 0.1-1 ram, and the width L is determined according to the width of the root of the eyelashes.

The glue 2 consists of the following components: mass percentage content: 38-48% isooctyl acrylate; 12-13% butyl methacrylate; 6-7% n-butyl acrylate; 0.9-1.5% methyl methacrylate; 0.08-0.2% ethylene glycol diacrylate; 31-42% water; 0.3-1% propylene glycol; 0.07-0.13% term-butyl peroxide; 0.05-0.15% sodium formaldehyde sulfoxylate; 0.05-0.1% sodium alkyl benzene sulfonate; 0.05-0.1% nonylphenol ethoxylate; 0.05-0.1% DCOIT; 0.1-0.2% ammonium persulfate; 0.05-0.15% sodium bicarbonate and 0.2-2% carbon black.

### Embodiment 5

This embodiment also proposes a production process of a convenient and quick-adhesive eyelashes. The types of grafting eyelashes 1 used include single eyelashes, multiple eyelashes, single cluster eyelashes, hot-melt eyelashes at the root, and glued eyelashes at the root, single eyelashes, A-type eyelashes, lower eyelashes.

### Embodiment 6

This embodiment also proposes a production process of a convenient and quick-adhesive eyelashes, after applying a certain amount of glue 2, let it dry at room temperature for 5 minutes-2 hours before packaging and sterilization and leave the factory for use. It can be taken out and worn directly from the packaging box, no need to apply glue, and no need to wait any longer. The false eyelashes obtained in this way can be worn faster, easier, safer, cheaper and more convenient in any place.

The above are all preferred embodiments of the present application, and are not intended to limit the protection scope of the present application. Therefore: all equivalent changes made according to the structure, shape and principle of the present application should be covered within the scope of the present application.

What is claimed is:

1. A production process of a convenient and quick-adhesive eyelashes, comprising the following steps:
applying a certain amount of glue to front or back of the root of the grafting eyelashes in direction from root to tip of the hair,
the grafting eyelashes are a single pair of false eyelashes or a single pair of lower eyelashes, and a certain amount of glue is applied on root eyelash stems of the single pair of false eyelashes or the single pair of lower eyelashes, and an applied glue height W is 1-2 mm, and a thickness H is 0.1-1 mm, a width L is determined according to a width of the root of the eyelashes;
the glue consists of the following components: mass percentage content: 37.8-48% isooctyl acrylate; 11.9-13% butyl methacrylate; 6-7% n-butyl acrylate; 0.9-1.5©% methyl methacrylate; 0.08-0.2% ethylene glycol diacrylate; 31-42% water; 0.3-1% propylene glycol; 0.07-0.13% tert-butyl peroxide; 0.05-0.15% sodium formaldehyde sulfoxylate; 0.05-0.1% sodium alkyl benzene sulfonate; 0.05-0.1% nonylphenol ethoxylate; 0.04-0.1% DCOIT; 0.1-0.2% ammonium persulfate; 0.05-0.15% sodium bicarbonate and 0.2-2% carbon black.

2. The production process of a convenient and quick-adhesive eyelashes according to claim 1, wherein the types of the grafting eyelashes include single eyelashes, multiple eyelashes, single cluster eyelashes, hot-melt eyelashes at the root, glued eyelashes at the root, segmented eyelashes, single eyelashes, A-type eyelashes, fishtail eyelashes, lower eyelashes, single pair of eyelashes, single pair of lower eyelashes.

3. The production process of a convenient and quick-adhesive eyelashes according to claim 2, wherein the material of the grafting eyelashes is animal hair, human hair, degradable silk, chemical fiber silk or protein silk.

4. The production process of a convenient and quick-adhesive eyelashes according to claim 1, wherein the glue consists of the following components: mass percentage content; 37.8-38.3% isooctyl acrylate; 11.9-12.4% butyl methacrylate; 6.1-6.6% n-butyl acrylate; 0.9-1.3% methyl methacrylate; 0.08-0.17% ethylene glycol diacrylate; 41.4-41.9% water; 0.3-0.8% propylene glycol; 0.07-0.13% tert-butyl peroxide; 0.05-0.15% sodium formaldehyde sulfoxylate; 0.05-0.1% sodium alkyl benzene sulfonate; 0.05-0.1% nonylphenol ethoxylate; 0.04-0.09% DCOIT; 0.1-0.2% ammonium persulfate; 0.05-0.15% sodium bicarbonate and 0.2-2% carbon black.

5. The production process of a convenient and quick-adhesive eyelashes according to claim 1, wherein the glue consists of the following components: mass percentage content: 38-48% isooctyl acrylate; 12-13% butyl methacrylate; 6-7% n-butyl acrylate; 0.9-1.5% methyl methacrylate; 0.08-0.2% ethylene glycol diacrylate; 31-42% water; 0.3-1% propylene glycol; 0.07-0.13% tert-butyl peroxide; 0.05-0.15% sodium formaldehyde sulfoxylate; 0.05-0.1% sodium alkyl benzene sulfonate; 0.05-0.1% nonylphenol ethoxylate; 0.05-0.1% DCOIT; 0.1-0.2% ammonium persulfate; 0.05-0.15% sodium bicarbonate and 0.2-2% carbon black.

6. A method of using the convenient and quick-adhesive eyelashes as claimed in claim 1, wherein after applying a certain amount of glue, let it dry at room temperature for 5 minutes-2 hours before packaging and sterilization and leave the factory for use.

7. The method according to claim 6, wherein a small brush or a glue dispenser tool is used to apply the glue.
